SEQUENCE LISTING (21 sequences)

SEQ ID NO: 1 - protein, 10 aa, Artificial Sequence
  VH CDR1
GFTFSNYGMH

SEQ ID NO: 2 - protein, 17 aa, Artificial Sequence
  VH CDR2
YISSGSSTIYYADSVKG

SEQ ID NO: 3 - protein, 9 aa, Artificial Sequence
  VH CDR3
ARRGLLLDY

SEQ ID NO: 4 - protein, 15 aa, Artificial Sequence
  VL CDR1
RASKTVSTSSYSYMH

SEQ ID NO: 5 - protein, 7 aa, Artificial Sequence
  VL CDR2
YASYLES

SEQ ID NO: 6 - protein, 9 aa, Artificial Sequence
  VL CDR3
QHSREFPWT

SEQ ID NO: 7 - protein, 116 aa, Artificial Sequence
  VH
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 8 - protein, 111 aa, Artificial Sequence
  VL
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 9 - protein, 446 aa, Artificial Sequence
  Full heavy chain of DX3
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, Artificial Sequence
  Fulll light chain of DX3
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 15 aa, Artificial Sequence
  Light chain CDR1 variant of DX3
RASKTVSTSSYGYMH

SEQ ID NO: 12 - protein, 10 aa, Artificial Sequence
  Heavy chain CDR1 variant of DX3
GFTFSNYGMN

SEQ ID NO: 13 - protein, 17 aa, Artificial Sequence
  Heavy chain CDR2 variant of DX3
YISNTSSTIYYADARKG

SEQ ID NO: 14 - protein, 111 aa, Artificial Sequence
  Light chain (VL) of DX3 variant 1
DIQMTHSPASLSASLGDRATITCRASKTVSTSSYSYMHWYQQKPGQPPKLLIKYASYLESGVPSRFSGSGSGTDYTLQINSLQPEDAATYYCQHSREFPWTFGGGTKVEIK

SEQ ID NO: 15 - protein, 111 aa, Artificial Sequence
  Light chain (VL) of DX3 variant 2
DIQMTQSPASLSASLGDRATITCRASKTVSTSSYSYMHWYQQKPGQPPKLLIKYASYLESGVPSRFSGSGSGTDYTLQINSLQPEDAANYYCQHSREFPWTFGGGTKVEIK

SEQ ID NO: 16 - protein, 111 aa, Artificial Sequence
  Light chain (VL) of DX3 variant 3
DIQMTHSPASLSASLGDRATITCRASKTVSTSSYGYMHWYQQKPGQPPKLLIKYASYLESGVPSRFSGSGSGTDYTLQINSLQPEDAANYYCQHSREFPWTFGQGTKVEIK

SEQ ID NO: 17 - protein, 116 aa, Artificial Sequence
  Heavy chain (VH) of DX3 variant 1
EVQLAESGGGLVQPGGSLRLSCAASGFTFSNYGMHWVRQAPGKGLEWVSYISSGSSTIYYADSVKGRYTISRENAKNSAYLQMNSLRAEDTGMYYCARRGLLLDYWGQGSNINISS

SEQ ID NO: 18 - protein, 116 aa, Artificial Sequence
  Heavy chain (VH) of DX3 variant 2
EVQLAESGGGLVQPGGSLRLSCAASGFTFSNYGMHWVRQAPGKGLEWVSYISSGSSTIYYADSVKGRYTISRENAKNSLYLQMNSLRAEDTGMYYCARRGLLLDYWGQGSNVTVSS

SEQ ID NO: 19 - protein, 116 aa, Artificial Sequence
  Heavy chain (VH) of DX3 variant 3
EVQLAESGGGLVQPGGSLRLSCAASGFTFSNYGMNWVRQAPGKGLEWVSYISNTSSTIYYADARKGRYTISRENAKNSAYLQMNSLRAEDTGMYYCARRGLLLDYWGQGSNINISS

SEQ ID NO: 20 - protein, 15 aa, Artificial Sequence
  Hinge sequence
EPKSCDKTHTCPPCP

SEQ ID NO: 21 - protein, 19 aa, Artificial Sequence
  Signal sequence
MEFGLSWVFLVALFRGVQC

(19) United States
(12) Patent Application Publication
(10) Pub. No.: US 2024/0226316 A1
(43) Pub. Date: Jul. 11, 2024

(54) Improved immunoglobulin I

(71) Applicant: Transmab Pty Ltd, South Melbourne, Victoria (AU)

(72) Inventors: Valentina DUBLJEVIC, South Melbourne, Victoria (AU); James CAMPBELL, South Melbourne, Victoria (AU)

(21) Appl. No.: 18/561,018

(22) PCT Filed: May 25, 2022
(86) PCT No.: PCT/AU2022/050503

§ 371 Date: Nov. 15, 2023

(30) Foreign Application Priority Data
May 25, 2021 (AU) 2021901567

(51) Int. Cl. A61K 47/68; A61K 49/00; A61K 51/10; A61P 35/00

(52) U.S. Cl. CPC A61K 47/6843; A61K 49/0002; A61K 51/1018; A61P 35/00

(57) ABSTRACT

The present disclosure relates to cell penetrating anti-DNA antibodies conjugated to a payload and methods of delivering these antibody conjugates to the brain. Compositions comprising these antibody conjugates may be useful for detecting and treating disease.

### FIELD OF THE INVENTION

The present disclosure relates to cell penetrating IgG anti-DNA antibodies conjugated to a payload and methods of delivering these antibody conjugates to the brain. Compositions comprising antibody conjugates of the disclosure may be useful for delivering payloads to the brain for detecting and treating disease.

### BACKGROUND OF THE INVENTION

The blood-brain barrier (BBB), a protective endothelial tissue surrounding the central nervous system (CNS), is a major impediment to the systemic delivery of high molecular weight therapeutic and diagnostic agents (e.g., antibodies) to the CNS. The BBB makes the development of new treatments for brain diseases or new neuroimaging agents challenging. New compositions and methods for delivering payloads to the brain are therefore required.

### SUMMARY OF THE INVENTION

Surprisingly, the present inventors have identified that payloads conjugated to a cell penetrating anti-DNA antibody can cross the blood brain barrier (BBB) and deliver payloads to the brain. Accordingly in a first aspect, the present disclosure relates to a method of delivering a payload to the brain of a subject, the method comprising administering to the subject a cell-penetrating, anti-DNA antibody conjugated to a payload, wherein the antibody is an IgG, humanized and comprises:
a heavy chain variable region (V_{H}) having a complementarity determining region (CDR) 1 as shown in SEQ ID NO: 1 or SEQ ID NO: 12, a CDR2 as shown in SEQ ID NO:2 or SEQ ID NO: 13 and a CDR3 as shown in SEQ ID NO: 3; and
a light chain variable region (V_{L}) having a CDR1 as shown in SEQ ID NO: 4 or SEQ ID NO: 11, a CDR2 as shown in SEQ ID NO: 5 and a CDR3 as shown in SEQ ID NO: 6. In one example, the subject is a human. In an example, the subject does not have brain cancer. In an example, the subject does not have a glioblastoma. In an example, the subject has a neurological disorder, a psychiatric disorder or a neurodegenerative disorder.

In another example, the subject has an intact blood brain barrier. Accordingly, in another example, the present disclosure encompasses a method of delivering a payload to the brain of a human subject, the method comprising administering to the subject a cell-penetrating, anti-DNA antibody conjugated to a payload, wherein the antibody is an IgG, humanized and comprises:
a heavy chain variable region (V_{H}) having a complementarity determining region (CDR) 1 as shown in SEQ ID NO: 1 or SEQ ID NO: 12, a CDR2 as shown in SEQ ID NO:2 or SEQ ID NO: 13 and a CDR3 as shown in SEQ ID NO: 3; and
a light chain variable region (V_{L}) having a CDR1 as shown in SEQ ID NO: 4 or SEQ ID NO: 11, a CDR2 as shown in SEQ ID NO: 5 and a CDR3 as shown in SEQ ID NO: 6 and wherein the subject has an intact blood brain barrier.

In an example, the antibody comprises a heavy chain variable region (V_{H}) having a complementarity determining region (CDR) 1 as shown in SEQ ID NO: 1, a CDR2 as shown in SEQ ID NO:2 and a CDR3 as shown in SEQ ID NO: 3; and a light chain variable region (V_{L}) having a CDR1 as shown in SEQ ID NO: 4, a CDR2 as shown in SEQ ID NO: 5 and a CDR3 as shown in SEQ ID NO: 6.

In another example, the antibody comprises:
(i) a V_{H} comprising a sequence at least 90% identical to the sequence as shown in SEQ ID NO: 7 or SEQ ID NO: 17 or SEQ ID NO: 18 or SEQ ID NO: 19 and, a V_{L} comprising a sequence at least 90% identical to the sequence as shown in SEQ ID NO: 8 or SEQ ID NO: 14 or SEQ NO: 15 or SEQ ID NO: 16; or,
(ii) a V_{H} comprising a sequence at least 90% identical to the sequence as shown in SEQ ID NO: 7 and, a V_{L} comprising a sequence at least 90% identical to the sequence as shown in SEQ ID NO: 8.

In another example, the antibody comprises a heavy chain comprising a sequence set forth in SEQ ID NO: 9 and a light chain comprising a sequence set forth in SEQ ID NO: 10.

The present inventors have also developed compositions comprising a cell-penetrating IgG anti-DNA antibody conjugated to a payload. Such compositions are particularly advantageous as they can cross the BBB and deliver payload to the brain. Accordingly, in another example, the present disclosure relates to a cell-penetrating anti-DNA antibody conjugated to a payload, wherein the antibody is an IgG, and comprises a V_{H} having a CDR1 as shown in SEQ ID NO: 1 or SEQ ID NO: 12, a CDR2 as shown in SEQ ID NO:2 or SEQ ID NO: 13 and a CDR3 as shown in SEQ ID NO: 3; and a V_{L} having a CDR1 as shown in SEQ ID NO: 4 or SEQ ID NO: 11, a CDR2 as shown in SEQ ID NO: 5 and a CDR3 as shown in SEQ ID NO: 6. In an example, the antibody is humanized. In an example, the antibody is provided in a pharmaceutical composition.

In an example, the pharmaceutical composition comprises a cell-penetrating anti-DNA antibody conjugated to a payload, wherein the antibody is an IgG, and comprises a V_{H} having a CDR1 as shown in SEQ ID NO: 1, a CDR2 as shown in SEQ ID NO:3 or SEQ ID NO: 13 and a CDR3 as shown in SEQ ID NO: 5; and a V_{L} having a CDR1 as shown in SEQ ID NO: 4 or SEQ ID NO: 11, a CDR2 as shown in SEQ ID NO: 5 and a CDR3 as shown in SEQ ID NO: 6. In an example, the antibody is humanized.

In an example, the antibody comprises:
(i) a V_{H} comprising a sequence as shown in SEQ ID NO:7 or SEQ ID NO: 17 or SEQ ID NO: 18 or SEQ ID NO: 19 and a V_{L} comprising a sequence as shown in SEQ ID NO: 8 or SEQ ID NO: 14 or SEQ NO: 15 or SEQ ID NO: 16; or
(ii) a V_{H} comprising a sequence as shown in SEQ ID NO:7 and a V_{L} comprising a sequence as shown in SEQ ID NO: 8. Accordingly, in another example, the antibody the antibody comprises a V_{H} comprising a sequence as shown in SEQ ID NO:7 and a V_{L} comprising a sequence as shown in SEQ ID NO: 8.

In an example, the antibody comprises a heavy chain variable region (V_{H}) having a complementarity determining region (CDR) 1 as shown in SEQ ID NO: 1, a CDR2 as shown in SEQ ID NO:2 and a CDR3 as shown in SEQ ID NO: 3; and a light chain variable region (V_{L}) having a CDR1 as shown in SEQ ID NO: 4, a CDR2 as shown in SEQ ID NO: 5 and a CDR3 as shown in SEQ ID NO: 6.

In an example, the payload is a therapeutic payload. In an example, the payload is a nucleic acid.

In an example, the therapeutic payload treats a condition affecting the brain of the subject. In an example, the condition is neurological disorder, a psychiatric disorder or a neurodegenerative disorder.

In an example, the therapeutic payload is selected from the group consisting of: an antibody, a nucleic acid, an anti-psychotic, an anti-depressant, an anti-inflammatory, an anti-neurodegenerative disease agent, a mood-stabilizer and an immunosuppressive agent. In an example, the antibody is provided as a bispecific antibody.

In an example, the payload is an imaging agent. In an example, the payload is a diagnostic agent. In an example, the payload is radiolabelled. In another example, the payload is fluorescently labelled.

In an example, the diagnostic agent is selected from the group consisting of: a radio pharmaceutical, an imaging agent and a nanoparticle.

In view of the present inventors surprising findings, the present disclosure, in an example, encompasses methods of treating a condition affecting the brain of a subject. Accordingly in an example, the present disclosure relates to a method of treating a condition affecting the brain of a human subject, the method comprising administering to the subject an antibody or composition disclosed herein. In an example, the subject does not have cancer. In another example, the subject has an intact blood brain barrier. In an example, the subject does not have brain cancer. In an example, the brain cancer is glioblastoma. In an example, the subject's blood brain barrier is intact.

In an example, when performing the methods of the disclosure, the antibody is provided as an antibody-drug conjugate.

### BRIEF DESCRIPTION OF THE ACCOMPANYING DRAWINGS

FIG. 1. 1A shows the structure of dimeric scFv and 1B shows a figure the structure of an intact antibody.

FIG. 2. Illustrates capacity of molecules to cross the BBB and deliver payload to the brain (% ID/cc). A region of interest (ROI) was drawn over the brain and the results were normalized to volume of the ROI. ¹²⁵I-Control IgG (n=2); ¹²⁵I-DX1 (n=3); ¹²⁵I-DX3 (n=3). Statistics: Unpaired t-test (72 and 96 h timepoints) or Two-way ANOVA (Dunnett's post hoc), * p≤0.05, ** p≤0.01. *** p≤0.001. **** p≤0.0001. Data Is presented as mean+SEM.

FIG. 3. Graphs illustrating the % ID/cc of molecules in the brain, thyroid, lungs, kidneys and liver. Regions of interest (ROIs) were drawn over kidney, liver, lungs, thyroid and brain. Results are normalized to ROI volume and can be considered as radioactivity concentration in specific tissue type.

FIG. 4. Graphs illustrating the radioactivity uptake as a percentage of the injected dose (% ID). Regions of interest (ROIs) were drawn over kidney, liver, lungs, thyroid and brain. Results are normalized only to injected dose of radioactivity. Different tissue types cannot be compared because of ROI volumes are different.

FIG. 5. Graph showing ex-vivo imaging from brain of non-tumor athymic nude mice and athymic nude mice bearing U87 MG orthotopic tumor.

### KEY TO SEQUENCE LISTING

SEQ ID NO: 1—Heavy chain CDR1 of DX3
SEQ ID NO: 2—Heavy chain CDR2 of DX3
SEQ ID NO: 3—Heavy chain CDR3 of DX3
SEQ ID NO: 4—Light chain CDR1 of DX3
SEQ ID NO: 5—Light chain CDR2 of DX3
SEQ ID NO: 6—Light chain CDR3 of DX3
SEQ ID NO: 7—Heavy chain (V_{H}) of DX3
SEQ ID NO: 8—Light chain (V_{L}) of DX3
SEQ ID NO: 9—Full Heavy chain of DX3
SEQ ID NO: 10—Full Light chain of DX3
SEQ ID NO: 11—Light chain CDR1 variant of DX3
SEQ ID NO: 12—Heavy chain CDR1 variant of DX3
SEQ ID NO: 13—Heavy chain CDR2 variant of DX3
SEQ ID NO: 14—Light chain (V_{L}) of DX3 variant 1
SEQ ID NO: 15—Light chain (V_{L}) of DX3 variant 2
SEQ ID NO: 16—Light chain (V_{L}) of DX3 variant 3
SEQ ID NO: 17—Heavy chain (V_{H}) of DX3 variant 1
SEQ ID NO: 18—Heavy chain (V_{H}) of DX3 variant 2
SEQ ID NO: 19—Heavy chain (V_{H}) of DX3 variant 3
SEQ ID NO: 20—Hinge sequence
SEQ ID NO: 21—Signal sequence

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., molecular biology, biochemistry, antibodies, antibody fragments such as single chain fragment variable and clinical studies).

The term “cell-penetrating” is used in the context of the present disclosure to refer to an anti-DNA antibody that is transported into the nucleus of living mammalian cells and binds DNA (e.g., single-stranded and/or double-stranded DNA). In an example, cell-penetrating antibodies disclosed herein need not necessarily maintain their cell penetrating properties after conjugation to a payload so long as they are capable of delivering the conjugated payload across the BBB.

The term “anti-DNA antibody” is used in the context of the present disclosure to refer to antibodies capable of binding DNA. In an example, anti-DNA binding antibodies disclosed herein need not necessarily maintain their DNA binding capabilities after conjugation to a payload so long as they are capable of delivering the conjugated payload across the BBB.

The term “antibody” is used in the context of the present disclosure to refer to immunoglobulin molecules immunologically reactive with a particular antigen and includes both polyclonal and monoclonal antibodies. The term also includes genetically engineered forms such as chimeric antibodies (e.g., humanized murine antibodies)

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. In an example, whole antibodies include an Fc region. The constant domains may be wild-type sequence constant domains (e.g., human wild-type sequence constant domains) or amino acid sequence variants thereof. In an example, the antibody is an IgG.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system” or “Kabat”.

Other conventions that include corrections or alternate numbering systems for variable domains include IMGT (Lefranc, et al. (2003), Dev Comp Immunol 27: 55-77), Chothia (Chothia C, Lesk A M (1987), J Mal Biol 196: 901-917; Chothia, et al. (1989), Nature 342: 877-883) and AHo (Honegger A, Pluckthun A (2001) J Mol Biol 309: 657-670). For convenience, examples of binding proteins of the present disclosure may also be labelled according to IMGT.

“Framework regions” (Syn. FR) are those variable domain residues other than the CDR residues.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy CH1, a linker, a CH2 and a CH3. In a heavy chain, a constant region comprises a Fc. In a light chain, a constant region generally comprise one constant domain (a CL1). An exemplary hinge sequence is shown in SEQ ID NO: 20.

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Exemplary heavy chain constant regions are gamma 1 (IgG1), gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of γ, α or δ heavy chain comprises two constant domains.

The term “conjugated” is used in the context of the present disclosure to refer to antibodies of the present disclosure that are conjugated to another compound, e.g., therapeutic compound or a diagnostic compound. Accordingly, in one example, the antibodies of the present disclosure are “conjugated”. The nature of the conjugation is not particularly limited so long as it is sufficient for an antibody of the disclosure to cross the BBB and deliver conjugated payload to the brain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill of those practicing in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the term “binds” in reference to the interaction of an antibody and an antigen means that the interaction is dependent upon the presence of a particular structure (e.g., an antigenic determinant or epitope) on the antigen. For example, an antibody recognizes and binds to a specific antigen structure rather than to antigens generally. For example, if an antibody binds to epitope “A”, the presence of a molecule containing epitope “A” (or free, unlabeled “A”), in a reaction containing labeled “A” and the antibody, will reduce the amount of labeled “A” bound to the antibody.

As used herein, the term “specifically binds” shall be taken to mean that the binding interaction between the antibody and DNA is dependent on detection of the DNA by the antibody. Accordingly, the antibody preferentially binds or recognizes DNA even when present in a mixture of other molecules or organisms.

In one example, the antibody reacts or associates more frequently, more rapidly, with greater duration and/or with greater affinity with DNA than it does with alternative antigens or cells. It is also understood by reading this definition that, for example, the antibody that specifically binds to DNA may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person. For example, an antibody of the disclosure is contacted with DNA or an alternative antigen. Binding of the antibody to DNA or alternative antigen is then determined and the antibody that binds as set out above to the DNA rather than the alternative antigen is considered to specifically bind to DNA.

Antibodies according to the present disclosure and compositions comprising the same can be administered to a subject to treat various indications. Terms such as “subject”, “patient” or “individual” are terms that can, in context, be used interchangeably in the present disclosure. In an example, the subject is a mammal. The mammal may be a companion animal such as a dog or cat, or a livestock animal such as a horse or cow. In one example, the subject is a human. For example, the subject can be an adult. In another example, the subject can be a child. In another example, the subject can be an adolescent. In one example, the subject has a condition affecting the brain of the subject. For example, the subject may have a neurological disorder, a psychiatric disorder or a neurodegenerative disorder. Suitable examples of a condition affecting the brain of the subject include, but are not limited to, motor neurone disease, dementia such as Alzheimer's disease and Lewy body disease, Parkinson's disease, Huntington's disease, multiple sclerosis, amyotrophic lateral sclerosis, Batten disease, chronic traumatic encephalopathy, depression, anxiety disorders, schizophrenia, addictive behaviours, stroke and infectious disease. In one example, the subject does not have brain cancer. In one example, the brain cancer is glioblastoma. In another example, the subject does not have localized brain tissue damage. In another example, the subject has an intact blood brain barrier. In this example, the subject may have cancer so long as their BBB is intact. In another example, the subject is a subject that needs neuroimaging to diagnose a condition affecting the brain of the subject.

The “blood brain barrier” or “BBB” refers to the principal interface between blood and the interstitial fluid that bathes synaptic connections within the parenchyma of the brain. It prevents entry into the brain of most drugs from the blood. The BBB is formed by tight junctions within the brain capillary endothelial plasma membranes and creates an extremely tight barrier that restricts the transport of molecules into the brain, even molecules as small as urea, molecular weight of 60 Da. The BBB is composed of two membranes in series, the luminal and abluminal membranes of the brain capillary endothelium. Molecules in the circulation can gain access to the interstitial fluid of the brain through one of the two mechanisms: (1) lipid-mediated free diffusion through the BBB; or (2) carrier- or receptor-mediated transport through the BBB.

As used herein, the term “intact blood brain barrier” refers to a BBB which has not been compromised in integrity or a BBB that is not “leaky”. Thus, an “intact blood brain barrier” can also refer to a BBB that does not allow antibodies to penetrate. In one example, an intact blood brain barrier is a blood brain barrier that is not broken down and does not permit the entry of molecules from the blood to the brain that are usually excluded from entry. Thus, an intact blood brain barrier has selective permeability. In an example, an “intact blood brain barrier” can also refer to a BBB that does not allow an unconjugated antibody to cross the BBB into the brain.

In contrast, a leaky BBB is one that has become impaired and permissive to the entry of molecules that are usually excluded from entry via the above referenced mechanisms. In an example, a leaky BBB is characterised by a breakdown in the tight junctions within the brain capillary endothelial plasma membranes. Methods to determine BBB permeability are known in the art. For example, Csaba et al., 2021 Neuropathology and Applied Neurobiology, 47, 297-315 have described GPCR internalization as a means to determine BBB permeability and Nguyen et al., 2013 NeuroImage: Clinical, 2, 658-662 developed a method of estimating BBB permeability using first-pass perfusion data.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease are mitigated or eliminated.

As used herein, the term “prevention” includes providing prophylaxis with respect to occurrence or recurrence of a disease in an individual. An individual may be predisposed to or at risk of developing the disease or disease relapse but has not yet been diagnosed with the disease or the relapse.

The term “treatment” is used in the context of the present specification to refer to the medical management of a patient with the intent to cure, ameliorate or stabilize a disease, pathological condition, or disorder. The term “treatment” includes active treatment, that is, treatment directed specifically toward the improvement of a disease, pathological condition, or disorder, and also includes causal treatment, that is, treatment directed toward removal of the cause of the associated disease, pathological condition, or disorder. In addition, the term “treatment” includes palliative treatment, that is, treatment designed for the relief of symptoms rather than the curing of the disease, pathological condition, or disorder; prophylactic treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

An “effective amount” refers to at least an amount effective, at dosages and for periods of time necessary, to achieve the desired therapeutic, prophylactic, diagnostic or otherwise informative result. An effective amount can be provided in one or more administrations. In some examples of the present disclosure, the term “effective amount” is meant an amount necessary to effect treatment of a disease or condition described below. In an example, an effective amount is an amount effective to image the brain of a subject. The effective amount may vary according to the disease or condition to be treated and also according to the weight, age, racial background, sex, health and/or physical condition and other factors relevant to the subject being treated. Typically, the effective amount will fall within a relatively broad range (e.g. a “dosage” range) that can be determined through routine trial and experimentation by a medical practitioner. The effective amount can be administered in a single dose or in a dose repeated once or several times over a treatment period. It is understood that the specific dose level for any particular patient depends upon a variety of factors including the activity of the specific antibody employed, the age, body weight, general health, sex, diet, time of administration, route of administration, and rate of excretion, drug combination and the severity of the particular disease undergoing therapy.

A “therapeutically effective amount” is at least the minimum concentration required to effect a measurable improvement of a particular disorder (e.g. neurodegenerative disease or a neurological disorder). A therapeutically effective amount herein may vary according to factors such as the disease state, age, sex, and weight of the patient, and the ability of the antibody to elicit a desired response in the individual. A therapeutically effective amount is also one in which any toxic or detrimental effects of the antibody are outweighed by the therapeutically beneficial effects. In the case of neurodegenerative disease, the therapeutically effective amount of the antibody may inhibit (i.e., slow to some extent and, in some examples, stop) neurodegenerative disease and neuronal death, disease progression; and/or relieve to some extent one or more of the symptoms associated with neurodegenerative disease. For neurodegenerative therapy, efficacy in vivo can, for example, be measured by assessing the duration of survival, time to disease progression (TTP), the response rates (RR), duration of response, and/or quality of life.

### Cell Penetrating Anti-DNA Antibodies

The present disclosure relates to antibodies conjugated to payload, wherein the antibody conjugate is capable of crossing the BBB to deliver the payload the brain. In an example, the antibody binds DNA. In another example, the antibody is cell penetrating. In an example, the antibody binds DNA and is cell penetrating. In one example, the antibody is an autoantibody derived from a subject or an animal with an autoimmune disease. In an example, the autoantibody is derived from a subject with systemic lupus erythematous, or an animal model thereof. The term “derived” as used herein encompasses recombinant forms of an antibody of the disclosure producing recombinant techniques such as the methods discussed below. For example, a nucleic acid sequence encoding an autoantibody from a subject with systemic lupus erythematous, or an animal model thereof can be provided in a recombinant system to produce a recombinant form of the antibody.

In one example, an antibody according to the present disclosure comprises a heavy chain variable region (V_{H}) having a complementarity determining region (CDR) 1 as shown in SEQ ID NO: 1, a CDR2 as shown in SEQ ID NO:2 and a CDR3 as shown in SEQ ID NO: 3; and a light chain variable region (V_{L}) having a CDR1 as shown in SEQ ID NO: 4, a CDR2 as shown in SEQ ID NO: 5 and a CDR3 as shown in SEQ ID NO: 6. Accordingly, in an example, the present disclosure encompasses an antibody which comprises a heavy chain variable region (V_{H}) having a complementarity determining region (CDR) 1 as shown in SEQ ID NO: 1, a CDR2 as shown in SEQ ID NO:2 and a CDR3 as shown in SEQ ID NO: 3; and a light chain variable region (V_{L}) having a CDR1 as shown in SEQ ID NO: 4, a CDR2 as shown in SEQ ID NO: 5 and a CDR3 as shown in SEQ ID NO: 6, wherein the antibody is conjugated to a payload. Such antibody conjugates are particularly useful in that they can cross the BBB and deliver their payload to the brain of a subject. Accordingly, in an example, the present disclosure encompasses an antibody which comprises a heavy chain variable region (V_{H}) having a complementarity determining region (CDR) 1 as shown in SEQ ID NO: 1, a CDR2 as shown in SEQ ID NO:2 and a CDR3 as shown in SEQ ID NO: 3; and a light chain variable region (V_{L}) having a CDR1 as shown in SEQ ID NO: 4, a CDR2 as shown in SEQ ID NO: 5 and a CDR3 as shown in SEQ ID NO: 6, wherein the antibody is conjugated to a payload and, wherein, upon administration to a subject, the antibody crosses the blood brain barrier and delivers the payload to the brain of a subject.

The present inventors have also identified CDR variants of the above referenced example. Accordingly, in another example, the antibody comprises an V_{H} having a CDR1 as shown in SEQ ID NO: 1 or SEQ ID NO: 12, a CDR2 as shown in SEQ ID NO:2 or SEQ ID NO: 13 and a CDR3 as shown in SEQ ID NO: 3; and a light chain variable region (V_{L}) having a CDR1 as shown in SEQ ID NO: 4 or SEQ ID NO: 11, a CDR2 as shown in SEQ ID NO: 5 and a CDR3 as shown in SEQ ID NO: 6.

In another example, the antibody comprises a V_{H} having a CDR1 as shown in SEQ ID NO: 1, a CDR2 as shown in SEQ ID NO:2 and a CDR3 as shown in SEQ ID NO: 3; and a light chain variable region (V_{L}) having a CDR1 as shown in SEQ ID NO: 4, a CDR2 as shown in SEQ ID NO: 5 and a CDR3 as shown in SEQ ID NO: 6.

In another example, the antibody comprises a V_{H} having a CDR1 as shown in SEQ ID NO: 12, a CDR2 as shown in SEQ ID NO:13 and a CDR3 as shown in SEQ ID NO: 3; and a light chain variable region (V_{L}) having a CDR1 as shown in SEQ ID NO: 11, a CDR2 as shown in SEQ ID NO: 5 and a CDR3 as shown in SEQ ID NO: 6. In the above referenced examples, the CDRs are subject to at least one amino acid substitution. In another example, the CDRs are subject to at least two amino acid substitutions. In another example, the CDRs are subject to at least three amino acid substitutions. In an example, the substitution(s) are in CDR1. In another example, the substitution(s) are in V_{H} CDR1. In another example, the substitution(s) are in V_{L} CDR2. In another example, the substitution(s) are in V_{H} CDR2.

In another example, the antibody comprises a V_{H} comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 7.

In another example, the antibody comprises a V_{H} comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 17.

In another example, the antibody comprises a V_{H} comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 18.

In another example, the antibody comprises a V_{H} comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 19.

In another example, the antibody comprises a V_{H} comprising a sequence as shown in SEQ ID NO: 7. In another example, the antibody comprises a V_{H} comprising a sequence as shown in SEQ ID NO: 17. In another example, the antibody comprises a V_{H} comprising a sequence as shown in SEQ ID NO: 18. In another example, the antibody comprises a V_{H} comprising a sequence as shown in SEQ ID NO: 19.

In another example, the antibody comprises a V_{L} comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 8.

In another example, the antibody comprises a V_{L} comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 14.

In another example, the antibody comprises a V_{L} comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 15.

In another example, the antibody comprises a V_{L} comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 16.

In another example, the antibody comprises a V_{L} comprising a sequence as shown in SEQ ID NO: 8. In another example, the antibody comprises a V_{L} comprising a sequence as shown in SEQ ID NO: 14. In another example, the antibody comprises a V_{L} comprising a sequence as shown in SEQ ID NO: 15. In another example, the antibody comprises a V_{L} comprising a sequence as shown in SEQ ID NO: 16.

In another example, the antibody comprises a heavy chain comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 9.

In another example, the antibody comprises a heavy chain comprising a sequence at least 80%, or at least 85%, or at least 90%, or at least 95%, or at least 97%, or at least 98%, or at least 98.5%, or at least 99%, or at least 99.5%, or at least 99.6%, or at least 99.7%, or at least 99.8%, or at least 99.9% identical to the sequence as shown in SEQ ID NO: 10.

In another example, the IgG anti-DNA antibody according to the present disclosure comprises a heavy chain comprising a sequence set forth in SEQ ID NO: 9 and a light chain comprising a sequence set forth in SEQ ID NO: 10.

In an example, the antibody is an IgG.

In an example, the antibody is monoclonal. Monoclonal antibodies are one exemplary form of antibodies contemplated by the present disclosure. The term “monoclonal antibody” or “MAb” refers to a homogeneous antibody population capable of binding to the same antigen(s), for example, to the same epitope within the antigen. This term is not intended to be limited as regards to the source of the antibody or the manner in which it is made.

In an example, antibodies encompassed by the present disclosure may be “humanized”. In an example, the CDRs are humanized. A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. In an example, the humanized antibody will also comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)).

In an example, antibodies of the disclosure are used to deliver a payload to the brain of a subject.

In an example antibodies of the disclosure are provided as an antibody-drug conjugate. Such antibodies can be specifically designed to deliver payload to a target cell such as a cancer cell. Other examples of target cells include cells located in the brain of a subject that does not have brain cancer. Accordingly, in an example, the target cell is a neuron. In an example, the target cell is a glial cell. In an example, the target cell is a microglial cell. In another example, the target cell is a pericyte. In an example, the target cell is a brain epithelial cell.

### Antibody Production

### Recombinant Expression

In an example, the antibody is recombinant.

In the case of a recombinant antibody, a nucleic acid encoding the same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce immunoglobulin or antibody protein.

Suitable molecular cloning techniques are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells. Thus, another example of the disclosure provides an expression construct that comprises an isolated nucleic acid encoding an antibody of the disclosure and one or more additional nucleotide sequences. Suitably, the expression construct is in the form of, or comprises genetic components of, a plasmid, bacteriophage, a cosmid, a yeast or bacterial artificial chromosome as are understood in the art. Expression constructs may be suitable for maintenance and propagation of the isolated nucleic acid in bacteria or other host cells, for manipulation by recombinant DNA technology and/or for expression of the nucleic acid or an antibody of the disclosure.

Many vectors for expression in cells are available. The vector components generally include, but are not limited to, one or more of the following: a signal sequence (e.g. SEQ ID NO: 21), a sequence encoding the antibody (e.g., derived from the amino acid sequence information provided herein), an enhancer element, a promoter, and a transcription termination sequence. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin or antibody promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the antibody may be cultured in a variety of media, depending on the cell type used. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

The skilled artisan will understand from the foregoing description that the present disclosure also provides an isolated nucleic acid encoding an antibody of the present disclosure.

The present disclosure also provides an expression construct comprising an isolated nucleic acid of an antibody of the disclosure operably linked to a promoter. In one example, the expression construct is an expression vector.

In one example, the expression construct of the disclosure comprises a nucleic acid encoding a polypeptide (e.g., comprising a V_{H}) operably linked to a promoter and a nucleic acid encoding another polypeptide (e.g., comprising a V_{L}) operably linked to a promoter.

The disclosure also provides a host cell comprising an expression construct according to the present disclosure.

The present disclosure also provides an isolated cell expressing an antibody of the disclosure or a recombinant cell genetically-modified to express the antibody.

Methods for purifying antibodies according to the present disclosure are known in the art and/or described in WO2019/018426.

### Payloads/Conjugates

Antibodies of the present disclosure are conjugated to a payload. The antibody can be directly or indirectly bound to the payload (e.g., can comprise a linker in the case of indirect binding). In an example, the payload is a compound. In an example, the payload is a small molecule. In an example, the payload is a therapeutic. In an example, the payload is an imaging agent. In an example, the payload is a drug. In an example, the antibody is provided as an antibody-drug conjugate.

Examples of suitable payloads include but are not limited to, a radioisotope (e.g., iodine-125, iodine-131, yttrium-90 or indium-111), a detectable label (e.g., a fluorophore or a fluorescent nanocrystal or quantum dot), a therapeutic compound, a colloid (e.g., gold), a toxin (e.g., ricin or tetanus toxoid), a nucleic acid, a peptide (e.g., a serum albumin binding peptide), a protein (e.g., a protein comprising an antigen binding domain of an antibody or serum albumin), an agent that increases the half-life of the compound in a subject (e.g., polyethylene glycol or other water soluble polymer having this activity) and mixtures thereof.

In an example, the payload is a nucleic acid. Exemplary nucleic acids include DNA (e.g., complementary DNA (cDNA), genomic DNA (gDNA)), RNA (e.g., message RNA (mRNA), short hairpin RNA (shRNA), short inhibitory RNA (siRNA), ribosomal RNA (rRNA), tRNA, microRNA, DNA or RNA analogues (e.g., containing base analogues, sugar analogues and/or a non-native backbone and the like), RNA/DNA hybrids and polyamide nucleic acids (PNAs), all of which can be in single- or double-stranded form. In an example, the nucleic acid is isolated. As used herein, the term “isolated nucleic acid” means a nucleic acid that is altered or removed from the natural state through human intervention. Another exemplary nucleic acid is an oligonucleotide. The term “oligonucleotide” as used herein means a short DNA or RNA molecule. Oligonucleotides readily bind, in a sequence-specific manner, to their respective complementary oligonucleotides, DNA, or RNA to form duplexes. In an example, the oligonucleotides are inhibitory oligonucleotides. In an example, the term “inhibitory oligonucleotide” refers to any oligonucleotide that reduces the production, expression or biological activity of one or more proteins. For example, an inhibitory oligonucleotide can interfere with translation of mRNA into protein in a ribosome. In another example, an inhibitory oligonucleotide can be sufficiently complementary to either a gene or a mRNA encoding one or more proteins to bind to (hybridize with) a targeted gene(s) or mRNA thereby reducing expression or biological activity of the target protein. In another example, an inhibitory oligonucleotide inhibits the biological activity of an intracellular nucleic acid that does not code for a protein. For example, an inhibitory oligonucleotide can inhibit the biological activity of a non-coding RNA. Exemplary inhibitory oligonucleotides include isolated or synthetic antisense RNA or DNA, siRNA or siDNA, miRNA, miRNA mimics, shRNA or DNA and Chimeric Antisense DNA or RNA.

In an example, the payload is another antibody.

In one example, the payload is a therapeutic payload. Suitable therapeutic agents for use in the provided compositions and methods, e.g., for conjugation to the provided antibodies, include, but are not limited to, a therapeutic agent selected from the group consisting of analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, nucleic acids, antibodies and hormones. In one example, the therapeutic agent is selected from the group consisting of: an antibody, a nucleic acid, an anti-psychotic, an anti-depressant, an anti-inflammatory, an anti-neurodegenerative disease agent, a mood-stabilizer and an immunosuppressive agent. In one example, the therapeutic agent is an antibody. In an example, the antibody can be provided as a bispecific antibody. In one example, the therapeutic agent is a nucleic acid. The choice of agent and dosage can be determined readily by one of skill in the art based on the given disease being treated. In one example, the therapeutic payload treats a condition affecting the brain of the subject.

In an example, antibodies of the disclosure are provided as an antibody-drug conjugate. Antibody-drug conjugates can be targeted to a target cell, such as a brain cell or cancer cell. In an example, the drug is a cytotoxic agent. Cytotoxic agents include, but are not limited to, a chemotherapeutic agent, a radiotherapy agent, and an immunotoxin agent. In an example, the drug is a therapeutic agent. Examples of therapeutic agents are described above.

Those of skill in the art will appreciate that the nature of the therapeutic payload will be influenced by the indication being treated. Exemplary indications are discussed below.

In another example, the payload is a diagnostic agent. In one example, the diagnostic agent is selected from the group consisting of: a radio pharmaceutical, an imaging agent and a nanoparticle. In one example, the diagnostic agent is an imaging agent. Imaging agents and their use are known. Optionally, the imaging agent is a “detectable moiety,” which is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego. The detectable moiety can be selected from the group consisting of gamma-emitters, beta-emitters, and alpha-emitters, gamma-emitters, positron-emitters, X-ray-emitters and fluorescence-emitters. Suitable fluorescent compounds include fluorescein sodium, fluorescein isothiocyanate, phycoerythrin, and Texas Red sulfonyl chloride, Allophycocyanin (APC), Cy5-PE, CY7-APC, Alexa Fluor and Cascade yellow. In an example, the imaging agent is a radiolabelled payload. In another example, the imaging agent is a fluorescent payload. Again, those of skill in the art will appreciate that the nature of the diagnostic/imaging payload will be influenced by application.

The detectable moiety can be visualized using histochemical techniques, ELISA-like assays, confocal microscopy, fluorescent detection, cell sorting methods, nuclear magnetic resonance, radioimmunoscintigraphy, X-radiography, positron emission tomography, computerized axial tomography, magnetic resonance imaging, and ultrasonography.

Methods for attaching a drug or other small molecule pharmaceutical to an antibody are well known and can include use of bifunctional chemical linkers such as N-succinimidyl (4-iodoacetyl)-aminobenzoate; sulfosuccinimidyl(4-iodoacetyl)-aminobenzoate; 4-succinimidyl-oxycarbonyl-∀-(2-pyridyldithio) toluene; sulfosuccinimidyl-6-[α-methyl-∀-(pyridyldithiol)-toluamido] hexanoate; N-succinimidyl-3-(-2-pyridyldithio)-proprionate; succinimidyl-6-[3 (-(-2-pyridyldithio)-proprionamido] hexanoate; sulfosuccinimidyl-6-[3 (-(-2-pyridyldithio)-propionamido]hexanoate; 3-(2-pyridyldithio)-propionyl hydrazide, Ellman's reagent, dichlorotriazinic acid, S-(2-thiopyridyl)-L-cysteine, and the like. Further bifunctional linking molecules are discussed in, for example, U.S. Pat. Nos. 5,349,066, 5,618,528, 4,569,789, 4,952,394, and 5,137,877.

Antibody sequences can be coupled to active agents or carriers using methods known in the art, including but not limited to chemical conjugation, enzymatic conjugation, carbodiimide conjugation, esterification, sodium periodate oxidation followed by reductive alkylation, and glutaraldehyde crosslinking (Goldman et al. (1997) Cancer Res. 57:1447-1451; Cheng (1996) Hum. Gene Ther. 7:275-282; Neri et al. (1997) Nat. Biotechnol. 15:1271-1275; Nabel (1997) Vectors for Gene Therapy. In Current Protocols in Human Genetics, John Wiley & Sons, New York; Park et al. (1997) Adv. Pharmacol. 40:399-435; Pasqualini et al. (1997) Nat. Biotechnol. 15:542-546; Bauminger & Wilchek (1980) Meth. Enzymol. 70:151-159; U.S. Pat. No. 6,071,890; and European Patent No. 0439095). The conjugation may be via chemical means or enzymatic means.

The payload may be conjugated to amino acid residues, double sulfide bonds, glycans. For example, the payload may be conjugated to lysine residues, amine groups, cysteine residues or new engineered amino acids. In an example, the payload is conjugated to a Tyrosine. In an example, the payload is conjugated to a Lysine. In an example, the payload is conjugated to a Lysine and the antibodies affinity for DNA is reduced after conjugation. In an example, the payload is conjugated to a Lysine and the antibody does not substantially bind DNA after conjugation.

The linker can cleavable or noncleavable. Highly stable linkers can reduce the amount of payload that falls off in circulation, thus improving the safety profile, and ensuring that more of the payload arrives at the target cell. Linkers can be based on chemical motifs including disulfides, hydrazones or peptides (cleavable), or thioethers (noncleavable) and control the distribution and delivery of the active agent to the target cell. Cleavable and noncleavable types of linkers have been proven to be safe in preclinical and clinical trials (see, e.g., Brentuximab vedotin which includes an enzyme-sensitive linker cleavable by cathepsin; and Trastuzumab emtansine, which includes a stable, non-cleavable linker). In particular embodiments, the linker is a peptide linker cleavable by Edman degradation (Bąchor, et al., Molecular diversity, 17 (3): 605-11 (2013)).

A non-cleavable linker can keep the active agent within the cell or the target microenvironment. As a result, the entire antibody, linker and active agent enter the targeted cell where the antibody is degraded to the level of an amino acid. The resulting complex between the amino acid of the antibody, the linker and the active agent becomes the active drug. In contrast, cleavable linkers are catalyzed by enzymes in the target cell or microenvironment where it releases the active agent. Once cleaved, the payload can escape from the targeted cell and attack neighboring cells (also referred to as “bystander killing”). In the case of the disclosed binding proteins, cleavage of the linker can lead to two active agents, the antibody itself and its payload, which can have different mechanisms of action in the target cell or microenvironment.

In some embodiments, there is one or more additional molecules, between the active agent and the cleavage site. Other considerations include site-specific conjugation (TDCs) (Axup, Proceedings of the National Academy of Sciences, 109 (40): 16101-6 (2012) and conjugation techniques such as those described in Lyon, et al., Bioconjugate Chem., 32 (10): 1059-1062 (2014), and Kolodych, et al., Bioconjugate Chem., 26 (2): 197-200 (2015) which can improve stability and therapeutic index, and α emitting immunoconjugates (Wulbrand, et al., Multhoff, Gabriele, ed., PLoS ONE. 8 (5): e64730 (2013)).

In an example, the antibody is conjugated to nanoparticles or microparticles (for example as reviewed in Kogan et al., Nanomedicine (Lond). 2: 287-306, 2007). The nanoparticles may be metallic nanoparticles. The particles can be polymeric particles, liposomes, micelles, microbubbles, and other carriers and delivery vehicles known in the art.

If the delivery vehicle is a polymeric particle, the binding protein can be coupled directly to the particle or to an adaptor element such as a fatty acid which is incorporated into the polymer. Ligands may be attached to the surface of polymeric particles via a functional chemical group (carboxylic acids, aldehydes, amines, sulfhydryls and hydroxyls) present on the surface of the particle and present on the ligand to be attached. Functionality may be introduced post-particle preparation, by crosslinking of particles and ligands with homo- or heterobifunctional crosslinkers. This procedure may use a suitable chemistry and a class of crosslinkers (CDT, EDAC, glutaraldehydes, etc. as discussed in more detail below) or any other crosslinker that couples ligands to the particle surface via chemical modification of the particle surface after preparation.

Antibodies may also be attached to polymeric particles indirectly though adaptor elements which interact with the polymeric particle. Adaptor elements may be attached to polymeric particles in at least two ways. The first is during the preparation of the micro- and nanoparticles, for example, by incorporation of stabilizers with functional chemical groups during emulsion preparation of microparticles. For example, adaptor elements, such as fatty acids, hydrophobic or amphiphilic peptides and polypeptides can be inserted into the particles during emulsion preparation. In a second embodiment, adaptor elements may be amphiphilic molecules such as fatty acids or lipids which may be passively adsorbed and adhered to the particle surface, thereby introducing functional end groups for tethering to binding proteins. Adaptor elements may associate with micro- and nanoparticles through a variety of interactions including, but not limited to, hydrophobic interactions, electrostatic interactions and covalent coupling.

Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

Some exemplary compounds that can be conjugated to an antibody of the present disclosure are listed in Table 1.

TABLE 1
Compounds useful in conjugation.

| Group | Detail |
|---|---|
| Radioisotopes | ¹²³I, ¹²⁵I, ¹³⁰I, ¹³³I, ¹³⁵I, ⁴⁷Sc, ⁷²As, ⁷²Sc, ⁹⁰Y, ⁸⁸Y, |
| (either directly | ⁹⁷Ru, ¹⁰⁰Pd, ^{101m}Rh, ^{101m}Rh, ¹¹⁹Sb, ¹²⁸Ba, ¹⁹⁷Hg, |
| or indirectly) | ²¹¹At, ²¹²Bi, ¹⁵³Sm, ¹⁶⁹Eu, ²¹²Pb, ¹⁰⁹Pd, ¹¹¹In, ⁶⁷Gu, |
| | ⁶⁸Gu, ⁶⁷Cu, ⁷⁵Br, ⁷⁶Br, ⁷⁷Br, ^{99m}Tc, ¹¹C, ¹³N, ¹⁵O, ¹⁸I, |
| | ¹⁸⁸Rc, ²⁰³Pb, ⁶⁴Cu, ¹⁰⁵Rh, ¹⁹⁸Au, ¹⁹⁹Ag or ¹⁷⁷Lu |
| Half-life | Polyethylene glycol |
| extenders | Glycerol |
| | Glucose |
| Fluorescent | Phycoerythrin (PE) |
| probes | Allophycocyanin (APC) |
| | Alexa Fluor 488 |
| | Cy5.5 |
| Biologics | fluorescent proteins such as Renilla luciferase, GFP |
| | immune modulators or proteins, such as cytokines, e.g., |
| | an interferon |
| | toxins |
| | an immunoglobulin or antibody or antibody variable |
| | region |
| | half-life extenders such as albumin or antibody variable |
| | regions or peptides that bind to albumin |

In one aspect of the above examples, the antibody conjugates can be used to deliver conjugated payloads to the brain.

In an example, an antibody of the disclosure is not conjugated to a nanoparticle.

### Methods of Delivering a Payload

The methods of the present disclosure encompass delivering a payload to the brain of a subject such as a human subject. As used herein, the term “delivering” refers to the transfer of the payload across the tight junctions of the BBB from the blood to the brain. The amount of payload transferred may be measured as a percentage of the dose administered/volume of the brain (% ID/cc) or as a percentage of the dose administered. Thus, in one example, the amount of payload delivered is about 0.2%, or about 0.3%, or about 0.4%, or about 0.5%, or about 0.6%, or about 0.7%, or about 0.8%, or about 0.9% or about 1% of the dose administered to the subject. Accordingly in one example, the amount of payload delivered is about 0.4% of the dose administered to the subject. In another example, the amount of payload delivered is about 0.5% of the dose administered to the subject. In another example, the amount of payload delivered is between 0.3% and 0.6% of the dose administered to the subject. In another example, the amount of payload delivered is about 0.2% ID/cc, or about 0.3% ID/cc, about or 0.4% ID/cc, or about 0.5% ID/cc, or about 0.6% ID/cc, or about 0.7% ID/cc, or about 0.8% ID/cc, or about 0.9% ID/cc, or about 1% ID/cc, or about 1.2% ID/cc, or about 1.3% ID/cc, or about 1.4% ID/cc, or about 1.5% ID/cc, or about 1.6% ID/cc, or about 1.7% ID/cc, or about 1.8% ID/cc, or about 1.9% ID/cc, or about 2% ID/cc. Accordingly in one example, the amount of payload delivered is about 0.3% ID/cc. In another example, the amount of payload delivered is about 0.5% ID/cc of the dose administered to the subject. In another example, the amount of payload delivered is between 0.2% and 0.6% ID/cc of the dose administered to the subject.

The payload may be retained in the brain after administration. The amount of payload retained may be measured as a percentage of the dose administered/volume of the brain (% ID/cc) or as a percentage of the dose administered hours after administration. In one example, the payload may be retained in the brain for up to about 1 hour, or about 2 hours, or about 3 hours, or about 6 hours, or about 9 hours, or about 12 hours or about 15 hours, or about 18 hours, or about 21 hours, or about 24 hours, or about 30 hours, or about 36 hours, or about 42 hours, or about 48 hours, or about 60 hours, or about 72 hours or about 96 hours or about 120 hours after administration. In another example, the payload may be retained in the brain for up to about 1 day, or about 2 days, or about 3 days, or about 4 days, or about 5 days after administration. Accordingly in one example, the payload may be retained for up to 96 hours. In another example, the payload may be retained for up to 24 hours. In another example, the payload may be retained for up to 6 hours. In another example, the payload may be retained for up to 2 hours.

Delivery methods of the present disclosure encompass delivering a payload to the brain of a human subject by administering an antibody disclosed herein. For example, the methods of the disclosure can comprise administering to a subject a cell-penetrating, anti-DNA antibody conjugated to a payload, wherein the antibody comprises:
a heavy chain variable region (V_{H}) having a complementarity determining region (CDR) 1 as shown in SEQ ID NO: 1 (GFTFSNYGMH), a CDR2 as shown in SEQ ID NO:2 (YISSGSSTIYYADSVKG) and a CDR3 as shown in SEQ ID NO: 3 (ARRGLLLDY); and
a light chain variable region (V_{L}) having a CDR1 as shown in SEQ ID NO: 4 (RASKTVSTSSYSYMH), a CDR2 as shown in SEQ ID NO: 5 (YASYLES) and a CDR3 as shown in SEQ ID NO: 6 (QHSREFPWT). In another example, the subject has an intact blood brain barrier. In an example, the antibody is humanized. In an example, the antibody is an IgG. In an example, the tight junctions in the subjects BBB are intact. In an example, a subject BBB is intact when an antibody of the disclosure crosses the BBB more efficiently (e.g. in greater amount) relative to a control molecule (e.g. a molecule that is typically blocked from crossing the BBB). In an example, the subject does not have cancer. In an example, the subject does not have brain cancer. In these examples, the subject can be human. In an example, the integrity of a subjects BBB is assessed before administering an antibody or composition of the disclosure.

### Compositions

The present disclosure provides a pharmaceutical composition comprising an antibody of the disclosure conjugated to a payload. Various antibodies and payloads are discussed above. For example, the composition can comprises a cell-penetrating, anti-DNA antibody conjugated to a payload, wherein the antibody is an IgG, humanized and, comprises a V_{H} having a CDR1 as shown in SEQ ID NO: 1, a CDR2 as shown in SEQ ID NO: 2, a CDR3 as shown in SEQ ID NO: 3 and a V_{L} having a CDR1 as shown in SEQ ID NO: 4, a CDR2 as shown in SEQ ID NO: 5 and a CDR3 as shown in SEQ ID NO: 6.

In another example, the present disclosure provides a pharmaceutical composition comprising a cell-penetrating, anti-DNA antibody conjugated to a payload, wherein the antibody is an IgG, humanized and, comprises:
(i) a V_{H} comprising a sequence as shown in SEQ ID NO: 7; and
(ii) a V_{L} comprising a sequence as shown in SEQ ID NO: 8.

The compositions can also contain a pharmaceutically acceptable carrier or adjuvant for administration of the antibody. In some embodiments, the carrier is pharmaceutically acceptable for use in humans. The carrier or adjuvant should not itself induce the production of antibodies harmful to the individual receiving the composition and should not be toxic. Suitable carriers can be large, slowly metabolized macromolecules such as proteins, polypeptides, liposomes, polysaccharides, polylactic acids, polyglycolic acids, polymeric amino acids, ammo acid copolymers and inactive virus particles.

Pharmaceutically acceptable salts can be used, for example mineral acid salts, such as hydrochlorides, hydrobromides, phosphates and sulphates, or salts of organic acids, such as acetates, propionates, malonate and benzoates.

Pharmaceutically acceptable carriers in therapeutic compositions can additionally contain liquids such as water, saline, glycerol and ethanol. Additionally, auxiliary substances, such as wetting or emulsifying agents or pH buffering substances, can be present in such compositions.

The compositions of the presently disclosed subject matter can further comprise a carrier to facilitate composition preparation and administration. Any suitable delivery vehicle or carrier can be used, including but not limited to a microcapsule, for example a microsphere or a nanosphere (Manome et al. (1994) Cancer Res 54:5408-5413; Saltzman & Fung (1997) Adv Drug Deliv Rev 26:209-230), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al. (1997) Cancer Res 57:1447-1451 and U.S. Pat. Nos. 4,551,482, 5,714,166, 5,510,103, 5,490,840, and 5,855,900), and a polysome (U.S. Pat. No. 5,922,545).

A composition of the present invention may comprise a pharmaceutical composition that includes a pharmaceutically acceptable carrier. Suitable formulations include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS in the range of 0.1 to 10 mg/ml, about 2.0 mg/ml; and/or mannitol or another sugar in the range of 10 to 100 mg/ml, in some embodiments about 30 mg/ml; and/or phosphate-buffered saline (PBS). Any other agents conventional in the art having regard to the type of formulation in question can be used. In some examples, the carrier is pharmaceutically acceptable. In some examples, the carrier is pharmaceutically acceptable for use in humans.

Compositions of the present disclosure can have a pH between 5.5 and 8.5, preferably between 6 and 8, and more preferably about 7. The pH can be maintained by the use of a buffer. The composition can be sterile and/or pyrogen free. The composition can be isotonic with respect to humans. Compositions of the presently disclosed subject matter can be supplied in hermetically-sealed containers.

The compositions can include an effective amount of one or more antibodies as described herein. In some embodiments, a pharmaceutical composition can comprise an amount that is sufficient to treat, ameliorate, or prevent a desired disease or condition, or to exhibit a detectable therapeutic effect. Therapeutic effects also include reduction in physical symptoms. The precise effective amount for any particular subject will depend upon their size and health, the nature and extent of the condition, and therapeutics or combination of therapeutics selected for administration. The effective amount for a given situation is determined by routine experimentation as practiced by one of ordinary skill in the art.

The compositions of the invention can be administered in a variety of unit dosage forms depending upon the method of administration. Dosages for typical antibody pharmaceutical compositions are well known to those of skill in the art. Such dosages are typically advisory in nature and are adjusted depending on the particular therapeutic context or patient tolerance. The amount antibody adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer, Science 249: 1527-1533, 1990.

Routes of administration include, but are not limited to, injection, subcutaneous, intramuscular, intravenous, intraarterial. In one example, the antibody is delivered through intravenous administration. In another example, the antibody is delivered through subcutaneous administration. In another example, the antibody is delivered through injection. In another example, the antibody is delivered through infusion.

The compositions can be administered in a single dose treatment or in multiple dose treatments on a schedule and over a time period appropriate to the age, weight and condition of the subject, the particular antibody formulation used, and the route of administration.

### Conditions to be Treated, Diagnosed or Monitored

The methods of the disclosure encompass treatment, prophylaxis, imaging and diagnosis or various conditions. In an example, the condition is a neurological disorder. Examples of neurological disorders include motor neurone disease, dementia such as Alzheimer's disease and Lewy body disease, Parkinson's disease, Huntington's disease, multiple sclerosis, amyotrophic lateral sclerosis, Batten disease, chronic traumatic encephalopathy, depression, anxiety disorders, schizophrenia, addictive behaviours, stroke and infectious disease. In an example, the neurological disorder is a dementia. In an example, the neurological disorder is Alzheimer's disease.

### Methods of Treatment and Methods of Prophylaxis

Various therapeutic and prophylactic applications are envisaged in view of the findings by the present inventors indicating that antibodies of the disclosure can cross the BBB and deliver payload to the brain. In an example, the present disclosure encompasses methods of treatment or prophylaxis which comprise administering antibodies or compositions described herein to a subject in need thereof. In an example, the subject has a neurological disorder.

Accordingly, in an example, the present disclosure provides a method of treating a condition affecting the brain of a human subject, the method comprising administering to the subject an antibody or composition disclosed herein, wherein the subject does not have cancer.

In another example, the present disclosure provides the use of an antibody or composition disclosed herein in the manufacture of a medicament for treating a condition affecting the brain of a subject, wherein the subject does not have cancer.

In another example, the present disclosure provides a method of treating a condition affecting the brain of a human subject, the method comprising administering to the subject an antibody or composition disclosed herein, wherein the subject has an intact blood brain barrier.

In another example, the present disclosure provides the use of an antibody or composition disclosed herein in the manufacture of a medicament for treating a condition affecting the brain of a subject, wherein the subject has an intact blood brain barrier.

In one example, the present disclosure provides a method of treating a condition affecting the brain of a human subject, comprising administering a cell-penetrating, anti-DNA antibody conjugated to a payload, wherein the antibody is an IgG, humanized and comprises:
a heavy chain variable region (V_{H}) having a complementarity determining region (CDR) 1 as shown in SEQ ID NO: 1, a CDR2 as shown in SEQ ID NO:2 and a CDR3 as shown in SEQ ID NO: 3; and
a light chain variable region (V_{L}) having a CDR1 as shown in SEQ ID NO: 4, a CDR2 as shown in SEQ ID NO: 5 and a CDR3 as shown in SEQ ID NO: 6 and wherein the subject does not have cancer.

In another example, the present disclosure provides a method of treating a condition affecting the brain of a human subject, comprising administering a cell-penetrating, IgG anti-DNA antibody conjugated to a payload, wherein the antibody is an IgG, humanized and comprises:
a heavy chain variable region (V_{H}) having a complementarity determining region (CDR) 1 as shown in SEQ ID NO: 1, a CDR2 as shown in SEQ ID NO:2 and a CDR3 as shown in SEQ ID NO: 3; and
a light chain variable region (V_{L}) having a CDR1 as shown in SEQ ID NO: 4, a CDR2 as shown in SEQ ID NO: 5 and a CDR3 as shown in SEQ ID NO: 6 and wherein the subject has an intact blood brain barrier.

In one example, the present disclosure provides a method of treating a condition affecting the brain of a human subject, comprising administering a cell-penetrating, anti-DNA antibody conjugated to a payload, wherein the antibody is an IgG, humanized, and comprises:
(i) a V_{H} comprising a sequence at least 90% identical to the sequence as shown in SEQ ID NO: 7; and
(ii) a V_{L} comprising a sequence at least 90% identical to the sequence as shown in SEQ ID NO: 8 and wherein the subject does not have cancer.

In another example, the present disclosure provides a method of treating a condition affecting the brain of a human subject, comprising administering a cell-penetrating, anti-DNA antibody conjugated to a payload, wherein the antibody is an IgG, humanized, and comprises:
(i) a V_{H} comprising a sequence at least 90% identical to the sequence as shown in SEQ ID NO: 7 and
(ii) a V_{L} comprising a sequence at least 90% identical to the sequence as shown in SEQ ID NO: 8 and wherein the subject has an intact blood brain barrier.

In another example, the present disclosure provides a method of treating a condition affecting the brain of a human subject, comprising administering a cell-penetrating, anti-DNA antibody conjugated to a payload, wherein the antibody is an IgG, humanized, and comprises a heavy chain comprising a sequence set forth in SEQ ID NO: 9 and a light chain comprising a sequence set forth in SEQ ID NO: 10 and wherein the subject does not have cancer.

In another example, the present disclosure provides a method of treating a condition affecting the brain of a human subject, comprising administering a cell-penetrating, anti-DNA antibody conjugated to a payload, wherein the antibody is an IgG, humanized, and comprises a heavy chain comprising a sequence set forth in SEQ ID NO: 9 and a light chain comprising a sequence set forth in SEQ ID NO: 10 and wherein the subject has an intact blood brain barrier.

In various examples, antibodies and compositions disclosed herein may be used in the manufacture of a medicament for treating a condition affecting the brain of a subject, wherein the subject does not have cancer.

### Imaging and Diagnosis

In an example, an antibody of the disclosure is utilized for detecting site or sites of neurodegenerative disease, tissue damage, injury, infection, or ischemia. Such methods typically including administering to a subject in need thereof an effective amount an antibody disclosed herein conjugated to a payload such as an agent that is detectable using diagnostic imaging or nuclear medicine techniques, and detecting the agent. The diagnostic imaging or nuclear medicine technique can be, for example, PET-CT, bone scan, MRI, CT, echocardiography, ultrasound, and x-ray. In an example, the imaging is informative for diagnosis and/or monitoring on a neurological condition disclosed herein.

### EXAMPLES

### Example 1—Anti-DNA Binding Proteins

DX1 and DX3 are anti-DNA binding proteins which are derived from 3E10 and humanized. DX1 and DX3 have a V_{H} sequence having a CDR1 as shown in SEQ ID NO: 9, a CDR2 as shown in SEQ ID NO: 10, a CDR3 as shown in SEQ ID NO: 11 and a sequence V_{L} having a CDR1 as shown in SEQ ID NO: 12, a CDR2 as shown in SEQ ID NO: 13 and a CDR3 as shown in SEQ ID NO: 14.

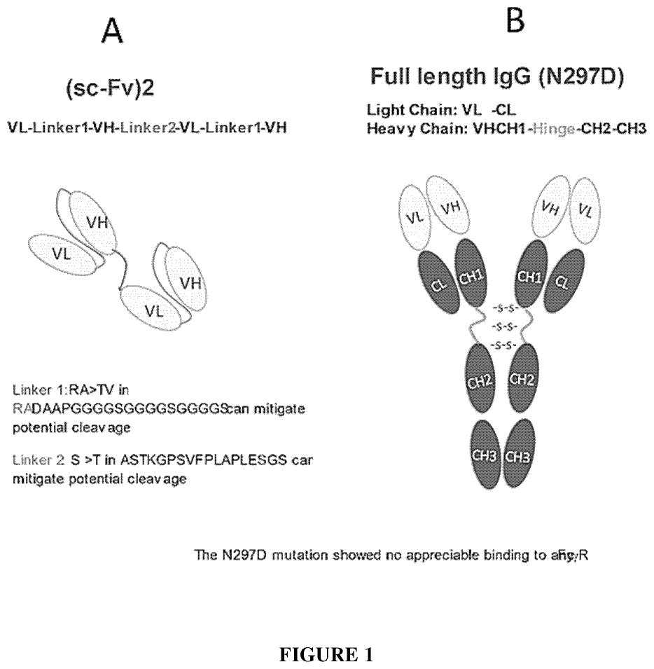

DX1 (FIG. 1A) is a (sc-FV)₂ molecule composed of two units each comprising a V_{L} domain linked to a V_{H} domain via a linker. DX3 is a full-length IgG molecule (FIG. 1B).

### Example 2—Radiolabelling

DX1, DX3 and a control IgG were radiolabelled with an Iodine-125 payload by conjugation to Tyrosine residues using Pierce pre-coated iodination tubes (Thermo Fisher Scientific) and following the manufacturer's instructions (“Direct method for Iodination”, MAN0016379 Rev. A.0, Thermo Scientific).

Briefly, the required radioactivity and the antibodies (for example 500 mCi (18.5 MBq)/100 μg protein, minimum of 1 mg/ml) were added to the iodination tube in neutral buffer and left to react for 15 min with occasional gentle mixing. A volume of 100-150 μl was used for optimal labelling. The reaction was stopped by removing the mixture from the iodination tube. Excess iodine was removed with centrifugal filtration Amicon® Ultra-4, 10 kDa cut-off (Sigma Alrich), followed by washing with PBS.

Depending on the yield, the final volume was adjusted to approximately 100 MBq/ml. Radiochemical purity was determined with instant thin layer chromatography using 85% MeOH as mobile phase. Radiolabelled protein has Rf=0. Additionally, samples of iodinated antibodies were also analyzed with radio-HPLC (Agilent Infinity II, 1260) with UV detector (280 nm) and Posiram radiodetector (Lablogic). Size exclusion column SEC-2000 (Phenomenex) was set to 30° C. and mobile phase was 0.1 M sodium phosphate pH 6.8. The labelled antibodies were analyzed for radiochemical purity and aggregation or breakdown.

### Labelling Yield

DX1 was 98.4% with 94.5% recovery from the purification. The calculated specific activity after labelling was 455.0 MBq/mg.
DX3 was 96.3% with 79.6% recovery from the purification. The calculated specific activity was 445.2 MBq/mg.
IgG control was 98.8% with 28.8% recovery from the purification. The calculated specific activity was 443.4 MBq/mg.

### Example 3—Evaluation of Payload Delivery to the Brain

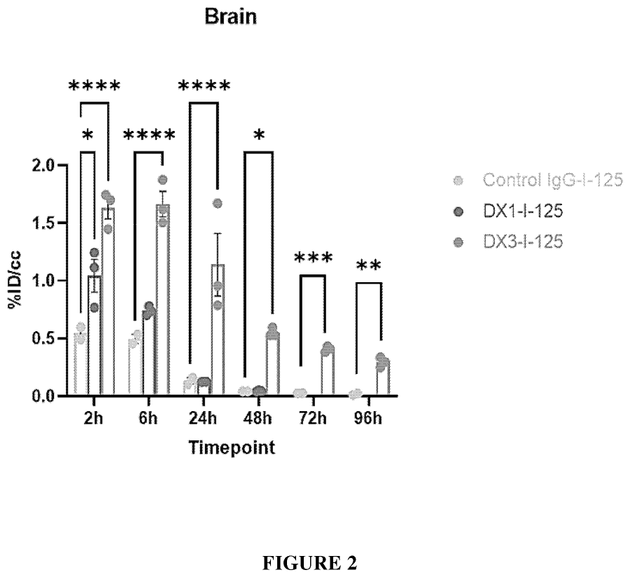

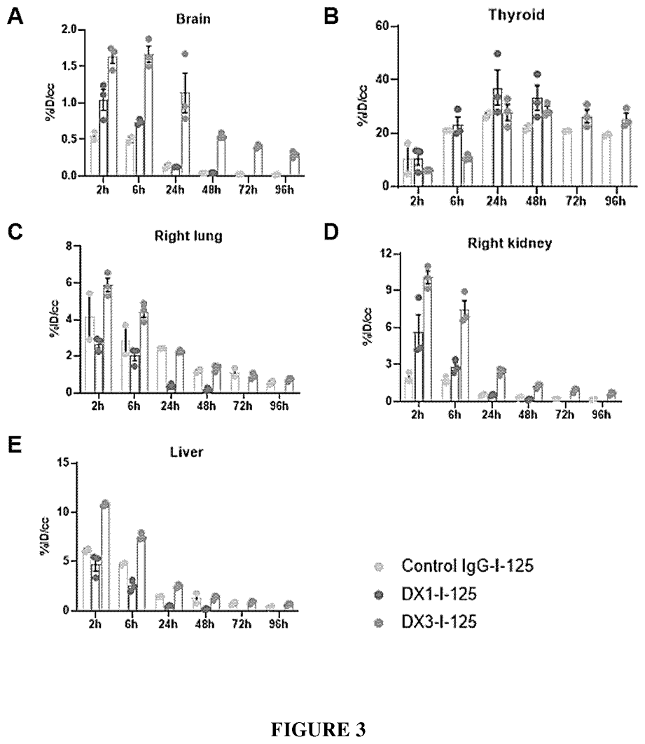

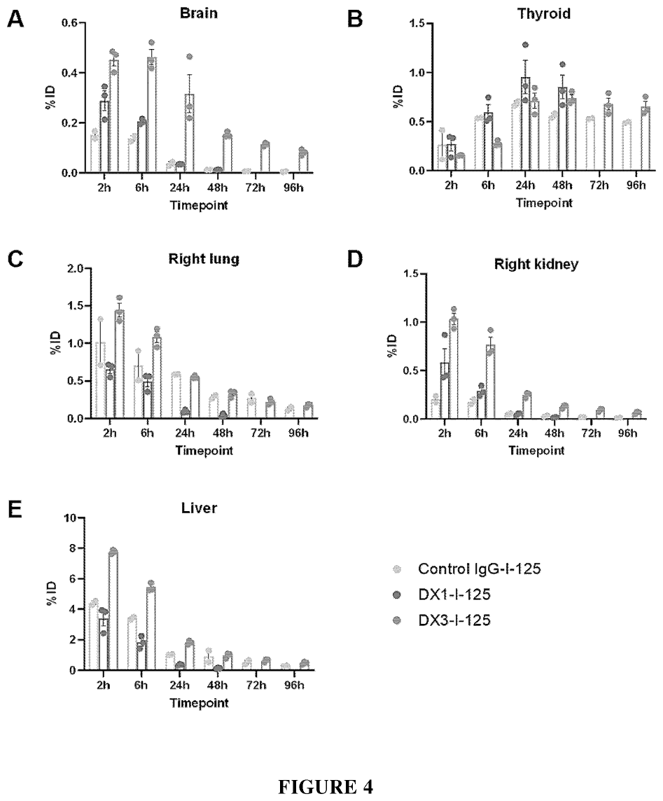

DX1, DX3 and IgG control were administered intravenously to healthy mice with an intact blood brain barrier and payload delivery to the brain was assessed in each instance (FIG. 2). Payload delivery to the brain was measured by monitoring radioactivity at 2 h, 6 h, 24 h, 48 h, 72 h and 96 h after administration. Payload delivery to the thyroid, left and right lung, left and right kidney and liver were also assessed at these time points (FIGS. 3 and 4).

Radioactivity uptake was measured by anesthetizing the animals with isoflurane (4-5% induction, 1.5-2% maintenance) with oxygen enriched air (3-600 mL/min) as a carrier gas. The animals were transferred to a SPECT/CT scanner (NanoSPECT/CT Plus, Mediso). Three mice were imaged simultaneously and 3D helical SPECT imaging was performed for the whole body over 60 min.

After the SPECT scan, helical CT was performed (180 projections, 55 kVp, 750 ms exposure time) from the same coordinates for anatomical reference. The temperature and breathing of the animals were monitored (SA Instruments Inc., NY, USA) and maintained at +37° C. and between 70-100 bpm respectively during the imaging process.

Radioactivity uptake in the brain, as percentage of injected dose per cubic centimeter (% ID/cc) was quantified using PMOD software v3.7 (PMOD technologies, Zurich, Switzerland). Regions of interest (ROIs) were drawn over kidney, liver, lungs and optionally other unexpected hotspot.

### Results

As shown in FIG. 2, both DX1 (2 h=1% ID/cc) and DX3 (2 h=1.6% ID/cc) were significantly more effective at delivering radiolabelled payload to the brain than control IgG (2 h=0.5% ID/cc). Surprisingly, despite its larger size, DX3 was significantly more effective at crossing the blood brain barrier and delivering radiolabelled payload to the brain than DX1 at the 2 hour time point. As shown in FIG. 2, DX3 delivered 1.5× more payload than DX1 suggesting that the IgG structure of DX3 significantly enhances payload delivery to the brain when compared with the smaller Fv structure of DX1. The improved payload delivery capability of DX3 was even more pronounced at the 24 hour time point, with 10× more DX3 being retained in the brain than either DX1 or IgG control.

The present inventors also unexpectedly identified that DX3 payload persists in the brain for much longer than both DX1 and IgG control payloads with increased radioactivity for DX3 being observed up to 96 hours after administration (FIG. 2). In contrast, minimal radioactivity was detected in the brain 48 h after administration of DX1 or IgG control (FIG. 2). This is a particularly advantageous finding because it suggests that the improved capability of DX3 to cross the blood brain barrier is also associated with prolonged bioavailability of payload in the brain.

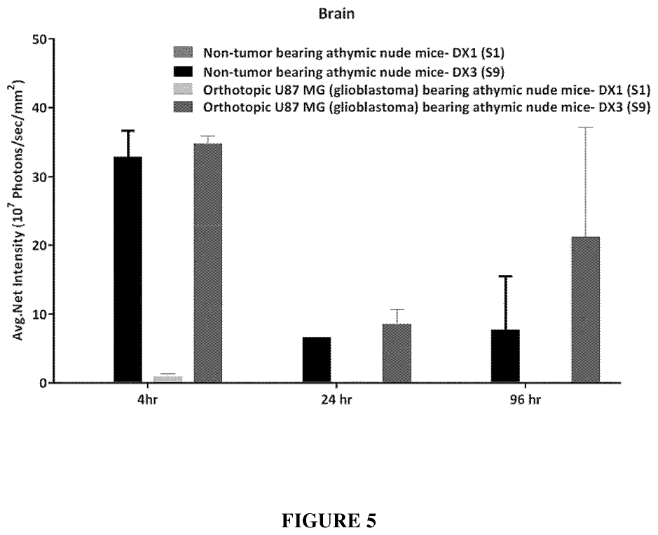

Interestingly, the improved payload delivery capabilities of both DX1 and DX3 relative to IgG control appeared to be restricted to the brain with no significant improvements in delivery being noted in the other organs assessed (FIGS. 4 and 5). These data further highlight the potential utility in using DX1 and DX3, to deliver payload(s) to the brain in view of their apparent preference to cross the blood brain barrier and target this organ, a characteristic not typically associated with antibodies.

### Example 4—Evaluation of Payload Delivery to the Brain (II)

The ability of Alexa647 labelled DX1 and Alexa647 labelled DX3 to cross the blood brain barrier of athymic nude mice and athymic nude mice bearing the orthotopic glioblastoma tumor mode was evaluated.

### Preparation of Tumour Cells

Cell line U87 MG was retrieved from Syngene In-vitro cell bank (Primary source-ATCC-U87 MG-HTB-14), propagated and used for cell injection with following details:

| | |
|---|---|
| Cell line | U87 MG |
| Origin/Type | Human glioblastoma |
| Passage No. | P17 and P18 |
| % Viability | ≥90% |
| Cell injection/animal | ~2 × 10⁶ cells/animal |

U87 MG (Human glioblastoma) cells with a viability of ≥90% was chosen for the study. Approximately 2×10⁶ cells suspended in 7 μL of PBS were used for cell injection per mouse. Athymic nude mice were intracranially injected with U87 MG cells (approximately 2×10⁶ cell per mouse).

### Intracranial Injection of Cells

Female athymic nude-Foxn1^{nu} mice housed in Individually Ventilated Cages (IVCs) were used for the study. U87 MG cell line were propagated into the animals by injecting the cells intracranially into the striatum (brain) of the animals. The intracerebral orthotropic glioblastoma injection was performed using a stereotaxic instrument. Athymic nude mice were anaesthetized using ketamine hydrochloride and xylazine (100 mg/kg+10 mg/kg; intraperitoneally). The depth of anaesthesia was ascertained by a toe pinch and the animal was placed in a stereotaxic frame. The surgical area was disinfected with povidone-iodine and sterilium and a 1-1.5 cm incision was made to expose the skull. The surface of the skull was cleaned and then swabbed with a dilute solution of hydrogen peroxide. A small burr hole was made 1 mm rostral to bregma (anterio-posterior) and 2 mm to the right (medio-lateral) using a microdrill. A Hamilton syringe containing U87 MG cells in 7 μL PBS was slowly lowered into the burr hole to the required depth (3 mm dorso-ventral). Cells were injected at a rate of 1 μL/minute using the stereotaxic injector and the syringe was left in place for an additional 4 minutes. After completion of injection, the site was gently cleaned, dried and bone wax was applied to the skull to close the burr hole. Skin was sutured with (Sutures—5 Metric—VICRYL SUTURE(POLYGLATIN 910), SIZE 5-0) and a thin layer of povidone-iodine ointment was applied on the skin. Animals were shifted to a recovery cage and allowed to wake up on heating pad. Animals were monitored for an additional 30 minutes and subsequently transferred to their home cage. In total, 26 mice were used for cell injection in four batches.

### Randomization

Non-tumor bearing healthy and active mice were selected for group I and II. Animals were randomized separately considering 7 mice (3+2+2) per group based on body weight (mean body weight ≈24 g). Similarly, U87 MG cell injected mice (n=26) were used for randomization for group III and IV. 14-19 days post cell injection, 14 mice were selected based on neurological scoring and randomized into two groups as orthotopic glioblastoma tumor bearing treatment groups (group III and IV) keeping 7 mice (3+2+2) per group based on body weight (mean body weight ≈24 g). Post randomization, dosing was initiated based on body weight as 10 mL/kg of dose volume.

### Dose Formulations and Route of Administration

The following dose formulations were used on the day of dosing.

| Test compounds | Quantity Received |
|---|---|
| Alexa fluor 647 | 26.03 mg in 12.5 mL |
| labelled DX1 (S1) | |
| Alexa fluor 647 | 11.66 mg in 2.2 mL |
| labelled DX3 (S9) | |

### Imaging

Post dosing, in vivo imaging using In-vivo Xtreme (Bruker) was carried at the following time points 0, 1, 4, 12, 24, 48, 72 & 96 h. At the end of the experimental period at 4 h (n=2), 24 h (n=2) & 96 h (n=3), blood withdrawal was carried out from selected animals and euthanized. The following tissues—Brain, liver, kidney, lung & spleen, skeletal muscle (eg. Gastrocnemius, Tibialis, Soleus-normal) were collected for ex vivo imaging and signal intensity was measured.

### Blood and Brain (PK Profiling)

At the end of the experimental period at 4 h (n=2), 24 h (n=2) & 96 h (n=3), blood withdrawal was carried out from selected animals. 40 μL of blood sample was mixed with 160 μL of HBSE (10 mM HEPES, 150 mM NaCl, 3 mM EDTA pH 7.4) solution, centrifuged at 4000 rpm for 15 min and supernatant was processed for PK profiling. Brain was harvested and snap frozen. Further PK analysis was performed on plasma and brain (snap frozen) samples.

### Results

In this imaging study, Alexa fluor 647 labelled test compounds-DX1 (S1) at a dose of 20 mg/kg and DX3 (S9) at a dose of 50 mg/kg; administered intravenously were evaluated for drug bio-distribution and crossing of Blood/Brain Barrier (BBB) in both normal athymic nude mice (non-tumor bearing) and athymic nude mice bearing U87 MG (Human Glioblastoma) tumors. The Alexa fluor 647 was conjugated to DX1 and DX3 via Lysine residues. Non-tumor bearing animals were randomized separately based on body weight into two groups group I and group II as 7 mice (3+2+2) in each group. Similarly, U87 MG orthotopic tumor bearing animals were selected based on neurological scoring (gait, coordination, exploratory behavior, limb strength, body position, reflexes and grooming) and randomized separately based on body weight (Mean body weight ≈24 g per group) into two groups—group III and IV as 7 mice (3+2+2) in each group.

A single dose of Alexa fluor 647 labelled DX1 (S1) (20 mg/kg) was administered intravenously to group I (Non-tumor bearing athymic nude mice—DX1) and group III (Orthotopic glioblastoma bearing nude mice—DX1). Similarly, a dose of 50 mg/kg of Alexa fluor 647 labelled DX3 (S9) was intravenously administered to group II (Non-tumor bearing athymic nude mice—DX3) and IV (Orthotopic glioblastoma bearing nude mice—DX3).

Imaging optimization was performed with empty vials and vials containing Alexa fluor labelled DX1 (S1) and DX3 (S9). Normal mice and mice bearing glioma were used for optimization. There was no background signal intensity observed at excitation: 650 nm; emission: 700 nm; 10 sec exposure. The same parameters were considered suitable and used for in vivo and ex vivo imaging. All animals were used for in vivo imaging from both dorsal and ventral side at each time point. After in vivo imaging at specific time points at 4 h (n=2), 24 h (n=2) and 96 h (n=3) animals were humanely euthanized and ex vivo imaging was performed for organs such as brain, kidney, liver, lungs, skeletal muscles and spleen.

Both the test compounds—Alexa fluor 647 labelled DX1 (S1) at 20 mg/kg and DX3 (S9) 50 mg/kg—administered intravenously were well tolerated when evaluated for bio-distribution and crossing for BBB in non-tumor bearing athymic nude mice and orthotopical U87 MG bearing athymic nude mice.

Ex vivo imaging (FIG. 5) shows that Alexa fluor 674 labelled DX3 can be detected in the brain of non-tumour bearing athymic nude mice and orthotopic U87 MG bearing athymic nude mice. This is a particularly advantageous finding because it suggests that DX3 is able to cross an intact blood brain barrier. Taken together with the above referenced findings, the present examples support a general concept for cell-penetrating, anti-DNA antibody mediated transport of various payloads across the BBB.

It will be appreciated by persons skilled in the art that numerous variations and/or modifications may be made to the disclosure as shown in the specific embodiments without departing from the spirit or scope of the disclosure as broadly described. The present embodiments are, therefore, to be considered in all respects as illustrative and not restrictive.

All publications discussed above are incorporated herein in their entirety. Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

The present application claims priority from AU2021901567 filed 25 May 2021, the disclosure of which are incorporated herein by reference.

1. A method of delivering a payload to the brain of a human subject, the method comprising administering to the subject a cell-penetrating, anti-DNA antibody conjugated to a payload, wherein the antibody is an IgG, humanized and comprises:
a heavy chain variable region (V_{H}) having a complementarity determining region (CDR) 1 as shown in SEQ ID NO: 1 or SEQ ID NO: 12, a CDR2 as shown in SEQ ID NO:2 or SEQ ID NO: 13 and a CDR3 as shown in SEQ ID NO: 3; and
a light chain variable region (V_{L}) having a CDR1 as shown in SEQ ID NO: 4 or SEQ ID NO: 11, a CDR2 as shown in SEQ ID NO: 5 and a CDR3 as shown in SEQ ID NO: 6 and wherein the subject does not have cancer.

2. A method of delivering a payload to the brain of a human subject, the method comprising administering to the subject a cell-penetrating, anti-DNA antibody conjugated to a payload, wherein the antibody is an IgG, humanized and comprises:
a heavy chain variable region (V_{H}) having a complementarity determining region (CDR) 1 as shown in SEQ ID NO: 1 or SEQ ID NO: 12, a CDR2 as shown in SEQ ID NO:2 or SEQ ID NO: 13 and a CDR3 as shown in SEQ ID NO: 3; and
a light chain variable region (V_{L}) having a CDR1 as shown in SEQ ID NO: 4 or SEQ ID NO: 11, a CDR2 as shown in SEQ ID NO: 5 and a CDR3 as shown in SEQ ID NO: 6 and wherein the subject has an intact blood brain barrier.

3. The method of any one of claim 1 or claim 2, wherein the antibody comprises:
(i) a V_{H} comprising a sequence at least 90% identical to the sequence as shown in SEQ ID NO: 7 or SEQ ID NO: 17 or SEQ ID NO: 18 or SEQ ID NO: 19 and, a V_{L} comprising a sequence at least 90% identical to the sequence as shown in SEQ ID NO: 8 or SEQ ID NO: 14 or SEQ NO: 15 or SEQ ID NO: 16; or,
(ii) a V_{H} comprising a sequence at least 90% identical to the sequence as shown in SEQ ID NO: 7 and, a V_{L} comprising a sequence at least 90% identical to the sequence as shown in SEQ ID NO: 8.

4. The method of claim 3, wherein the antibody comprises a heavy chain comprising a sequence set forth in SEQ ID NO: 9 and a light chain comprising a sequence set forth in SEQ ID NO: 10.

5. A pharmaceutical composition comprising a cell-penetrating anti-DNA antibody conjugated to a payload, wherein the antibody is an IgG, humanized and comprises a V_{H} having a CDR1 as shown in SEQ ID NO: 1 or SEQ ID NO: 12, a CDR2 as shown in SEQ ID NO: 2 or SEQ ID NO: 13 and a CDR3 as shown in SEQ ID NO: 3; and a V_{L} having a CDR1 as shown in SEQ ID NO: 4 or SEQ ID NO: 11, a CDR2 as shown in SEQ ID NO: 5 and a CDR3 as shown in SEQ ID NO: 6.

6. The pharmaceutical composition of claim 5, wherein the antibody comprises:
(i) a V_{H} comprising a sequence as shown in SEQ ID NO:7 or SEQ ID NO: 17 or SEQ ID NO: 18 or SEQ ID NO: 19 and a V_{L} comprising a sequence as shown in SEQ ID NO: 8 or SEQ ID NO: 14 or SEQ NO: 15 or SEQ ID NO: 16; or
(ii) a V_{H} comprising a sequence as shown in SEQ ID NO:7 and a V_{L} comprising a sequence as shown in SEQ ID NO: 8.

7. The method of any one of claims 1-4 or the composition of claim 5 or claim 6, wherein the antibody comprises a heavy chain variable region (V_{H}) having a complementarity determining region (CDR) 1 as shown in SEQ ID NO: 1, a CDR2 as shown in SEQ ID NO:2 and a CDR3 as shown in SEQ ID NO: 3; and a light chain variable region (V_{L}) having a CDR1 as shown in SEQ ID NO: 4, a CDR2 as shown in SEQ ID NO: 5 and a CDR3 as shown in SEQ ID NO: 6.

8. The method or the composition of claim 7, wherein the antibody comprises a V_{H} comprising a sequence as shown in SEQ ID NO: 7 and a V_{L} comprising a sequence as shown in SEQ ID NO: 8.

9. The method of any one of claims 1-4 or 7-8 or the composition of any one of claims 5-8, wherein the payload is a therapeutic payload.

10. The method or the composition of claim 9, wherein the therapeutic payload treats a condition affecting the brain of the subject.

11. The method or the composition of claim 10, wherein the condition is neurological disorder, a psychiatric disorder or a neurodegenerative disorder.

12. The method or the composition of any one of claims 9-11, wherein the therapeutic payload is selected from the group consisting of: a nucleic acid, an anti-psychotic, an anti-depressant, an anti-inflammatory, an anti-neurodegenerative disease agent, a mood-stabilizer and an immunosuppressive agent.

13. The method of any one of claims 1-4 or 7-8 or the composition of any one of claims 5-8, wherein the payload is a diagnostic agent.

14. The method or the composition of claim 13, wherein the diagnostic agent is selected from the group consisting of: a radio pharmaceutical, an imaging agent and a nanoparticle.

15. A method of treating a condition affecting the brain of a human subject, the method comprising administering to the subject the composition of any one of claims 7-12, wherein the subject does not have cancer.

16. Use of the composition of any one of claims 7-12 in the manufacture of a medicament for treating a condition affecting the brain of a subject, wherein the subject does not have cancer.

17. The method of any one of claims 1-4 or 7-12 or the use of claim 16, wherein the cancer is brain cancer.

18. The method or the use of claim 15, wherein the brain cancer is glioblastoma.

19. The method of any one of claims 1-4, 7-15 or 17-18 or the use of any one of claims 16-18, wherein the subject's blood brain barrier is intact.

20. The method of any one of claims 1-4 or 7-8 or the composition of any one of claims 5-8, wherein the payload is a nucleic acid.

21. The method of any one claims 1-4, 7-15 or 17-20, the composition of any one of claims 5-14 or 20, or the use of any one of claims 16-18, wherein the antibody is provided as an antibody-drug conjugate.
